(19) United States
(12) Patent Application Publication
(10) Pub. No.: US 2006/0165813 A1
(43) Pub. Date: Jul. 27, 2006

(54) Topical composition for treatment of skin disorders

(76) Inventors: Jerzy Hubert Edward Dangel, Sunnybank (AU); Romi Roy, Encinitas, CA (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/525,519

(22) PCT Filed: Sep. 2, 2003
(86) PCT No.: PCT/AU03/01124

§ 371 Date: Sep. 9, 2005

(30) Foreign Application Priority Data
Sep. 3, 2002 (US) 60407784
Nov. 27, 2002 (AU) 2002953357
Aug. 21, 2003 (AU) 2003904498

(51) Int. Cl. A61K 33/32; A61K 33/18

(52) U.S. Cl. 424/641; 424/666

(57) ABSTRACT

The present invention provides a topical composition comprising the combination of zinc ions, ammonium ions and chloride ions for treatment of skin disorders. The invention also relates to a method for treating or preventing a skin disorder using the topical composition of the present invention.

### FIELD OF THE INVENTION

The present invention relates to a topical composition for treatment of skin disorders. The invention also relates to a method for treating or preventing a skin disorder using the topical composition of the present invention.

### BACKGROUND OF THE INVENTION

With the increase in the world's ageing population there is a concomitant increase in occurrence of skin disorders, such as skin lesions and skin cancer, caused by extensive exposure of the skin to ultraviolet beta rays and chemicals in various cosmetic and skin care products.

Existing treatment of skin disorders, particularly actinic keratosis, includes topical photosensitised creams containing an amine compound. The amine compound absorbs light rays of a specific wavelength and are capable of penetrating the skin. The photosensitised creams have been demonstrated in clinical trials to be effective and well tolerated by patients (see for example Jeffes E W, J Dermatolog Treat 2002, 13 Supply 1:S19-23). Other treatments of skin disorders include, photodynamic therapy which involves the application of a heat impulse to localised sites of the skin for drug delivery into the stratum comeum. However, photodynamic therapy, though approved by the US FDA for clinical use, is not very practical and the concomitant continuous transport of drug molecules through the skin can result in systemic exposure to the drug molecules.

The migration of keratinocytes plays an important role in the re-epithelialisation of skin wounds, and zinc, copper and manganese have been shown to promote keratinocyte proliferation by modulating expression of integrins (see, for example, Tenaud e al 2000; Exp Dermatol 9(6):407-16). Matrix metalloproteinases (MMPs) are also believed to be involved in re-epithelialisation of skin wounds since they have been cloned from injured as well as intact skin keratinocytes (see for example, Lohl et al 2001, J Biol Chem 276(13) 10134-10144). All MMPs have two conserved domains, namely a catalytic domain which includes an Zn²⁺ binding site and a prodomain, and are bound to the cell membrane where they function either extracellulary or within the secretion pathway. The present inventors believe that the MMPs of keratinocytes are important regulators of skin tissue remodelling (including re-epithelialisation) through regulating skin cell migration. While not wishing to be bound by theory, it is accordingly considered that effective treatment of skin disorders may be achieved by promoting the activity of the MMPs of keratinocytes.

For reasons of convenience and specificity of action, it is desirable to formulate a composition for treatment of skin disorders as a topical composition which may be readily applied to localised sites of the skin. A problem which must be overcome by an effective topical composition is the natural barrier properties of skin which resists against the penetration of foreign substances. Existing methods of skin treatment developed to overcome the skin barrier include agents that disrupt the skin barrier. For instance, U.S. Pat. No. 6,190,894 reveals a method for disrupting the epithelial barrier function by topically applying to the skin a barrier-disrupting amount of a substance such as an inhibitor of fatty acid (eg ceramide) or phospholipid synthesis, a degradation enzyme or a stimulator of metabolic enzymes. Other skin treatments have involved the use of electrical current to promote penetration of active ingredients of a topical composition. For example, U.S. Pat. No. 5,195,953 relates to a method of increasing the absorption of an ionic form of a drug minoxidil through scalp epidermis with an electrical apparatus which provides an output of negative electrical current to cause vaso-dilation of the skin surface so to promote the penetration of the drug through the epidermis surface.

The present invention aims to provide a topical composition that is capable of efficient transcutaneous delivery of an active agent (ie Zn²⁺) to the dermis without the need to apply an external electric current or a chemical reducing agent. In addition, the present invention utilises small non-metal ions such as H⁺, Cl⁻ and NH₄⁺ to advantageously open up cell membrane channels to allow passage of small metal ions such as Zn²⁺.

The use of zinc has been previously proposed for topical compositions. For instance, U.S. Pat. No. 6,407,090 describes a method of treating apoptosis and protecting skin against harmful injurious agents such as oxidants, neurotoxins, and radiation, wherein the method involves the application of an effective amount of a zinc ionophore (ie a zinc pyrithione). However, zinc used in this formulation is in the form of a large molecule and may not therefore be capable of deeply penetrating the skin, thereby reducing the effectiveness of the zinc ionophore. Also, U.S. Pat. No. 5,928,659 teaches a method for treating skin lesions such as keratoses or distensae with a composition of unsaponifiable lipids from avocado fruit or seed emulsified with an aqueous phase consisting of very minute amounts of metal amino acid chelates, the metals being either zinc or copper, to enhance the efficiency of the active lipid composition. The fatty phase of the composition is 25-75%, avocado lipids make up 1 to 5% and the aqueous phase is between 0.1 to 2% of the total weight. Another example of a skin treatment composition containing small amounts of metals is described in U.S. Pat. No. 6,287,548. In this example, the composition comprises a synthetic mixture of salts which, when dissolved in a solvent such as water, is ionically composed of a mixture of sodium and magnesium cations and chloride and sulfate anions and the balance being water. However, the composition described in U.S. Pat. No. 6,287,548 is preferably free of added zinc, as it is taught that zinc has no therapeutic effect and may detract from the efficiency of the salt mixture. This is in contrast to the topical composition provided by the present invention.

### SUMMARY OF THE INVENTION

In a first aspect of the present invention, there is provided a topical composition for treatment of a skin disorder, the composition comprising a solution of:
(i) Zn²⁺ ions;
(ii) NH₄⁺ ions;
(iii) Cl⁻ ions; and, optionally,
(iv) a suitable excipient and/or carrier.

Preferably, the composition comprises a solution of zinc ammonium chloride and/or zinc chloride, in combination with ammonium chloride and hydrochloric acid.

Preferably, the present invention provides a topical composition for treatment of a skin disorder, the composition comprising a solution of:
(i) zinc ammonium chloride;
(ii) ammonium chloride;
(iii) hydrochloric acid; and, optionally,
(iv) a suitable excipient and/or carrier; or:
(i) zinc chloride;
(ii) ammonium chloride;
(iii) hydrochloric acid; and, optionally,
(iv) a suitable excipient and/or carrier.

In a second aspect of the present invention, there is provided a topical composition for treatment of a skin disorder, the composition consisting of an aqueous solution of:
(i) Zr²⁺ ions;
(ii) NH₄⁺ ions;
(iii) Cl⁻ ions; and, optionally,
(iv) a suitable excipient and/or carrier.

Preferably, the composition consists of a solution of zinc ammonium chloride and/or zinc chloride, in combination with ammonium chloride and hydrochloric acid.

Preferably, the invention provides a topical composition for treatment of a skin disorder, the composition consisting of an aqueous solution of:
(i) zinc ammonium chloride;
(ii) ammonium chloride;
(iii) hydrochloric acid; and, optionally,
(iv) a suitable excipient and/or carrier.

The composition includes zinc ammonium chloride preferably at a concentration in the range of about 30% to 75% by weight by volume of the composition, more preferably about 55% by weight by volume of the composition. The composition includes ammonium chloride preferably at a concentration in the range of about 5% to 20% by weight by volume of the composition, more preferably about 10% by weight by volume of the composition. The composition includes hydrochloric acid preferably at a concentration in the range of about 1% to 5% by weight by volume of the composition, more preferably about 3% to 5% by weight by volume of the composition.

Preferably, the present invention provides a topical composition for treatment of a skin disorder, the composition consisting of an aqueous solution of:
(i) zinc chloride;
(ii) ammonium chloride;
(iii) hydrochloric acid; and, optionally,
(iv) a suitable excipient and/or carrier.

The composition includes zinc chloride preferably at a concentration in the range of about 20% to 60% by weight by volume of the composition, more preferably about 30% to 45% by weight by volume of the composition. The composition includes ammonium chloride preferably at a concentration in the range of about 5% to 20% by weight by volume of the composition, more preferably about 8% to 10% by weight by volume of the composition. The composition includes hydrochloric acid preferably at a concentration in the range of about 1% to 5% by weight by volume of the composition, more preferably about 2% to 4% by weight by volume of the composition.

The carrier for use in the composition of the present invention is preferably water and, more preferably, deionised water. The water may comprise about 1 to 99%, more preferably 30 to 95%, and most preferably 90%, of the composition by weight by volume. The composition of the present invention preferably has a pH of no more than about 7. More preferably, the pH is in the range of about 2 to 5.

The composition of the present invention may include a suitable excipient selected from the group consisting of a fragrant agent, surfactant, stabiliser, dye, penetration enhancer and anti-oxidant. The composition is preferably in the form of a spray, aerosol, lotion, ointment, gel, cream or dispersion and the like.

In a third aspect of the present invention, there is provided a method for treating or preventing a skin disorder in a subject, the method comprising applying to the skin of the subject an effective amount of a composition according to the first or second aspect.

The skin disorder is preferably selected from the group consisting of skin lesions, hyperproliferative skin disorders, inflammatory skin disorders, actinic keratosis, solar lentigines, psoriasis, dermatitis, eczema, tenia, melanoma, basal cell carcinoma and squamous cell carcinoma.

### BRIEF DESCRIPTION OF THE ACCOMPANYING FIGURES

FIG. 1 shows a diagramatic representation of the mechanism of action of the topical composition of the present invention. On application of the composition on the surface of the damaged skin site a film is formed. There are ion channels present ubiquitously on various epithelial cells of the skin which are involved in proliferation, differentiation and cell volume regulation. The chloride ions from the solution initiate a conductive flow into the skin. The composition penetrates the hydrophilic regions of the stratum corneum surrounding the lipid-filled, damaged epithelial cells. The stratum corneum acts as a reservoir for the Z²⁺, NH₄⁺ and Cl⁻ ions. Due to the acidic nature of the composition a mixture of NH₃ and its protonated form NH₄⁺ fill up the reservoir. The apical surface of the epithelium is impermeable to NH₃, being a small molecule (17 DA), so NH₃ moves freely across the basolateral cell membranes making way for the NH₄⁺ ions to pass through. Basolateral exposure of the epithelial cells including stratum corneum to NH₃ and NH₄⁺ allows these ions to interact with the membrane proteins and ion channels structures making the metal ion transporter proteins accessible from the inner cell surface. The Zn²⁺ ion finally flows through the cell wall and binds to its receptors known as MMPs. MMPs are abundant on the inner cell surface in damaged epithelial cells such as in basal cell carcinomas. A relatively high concentration of zinc ammonium chloride or zinc chloride delivers maximum Zn²⁺ ions to the binding sites. Zn²⁺ ions bound to the MMPs within the damaged epithelial cells inhibits other cellular functions thereby disrupting cell-cell interaction at the interface between epidermis and the basement membrane while Zn²⁺ ions bound to the MMPs of normal keratinocytes promotes re-epithelialisation. The result is that the upper layers of the damaged stratum comeum subsequently “disconnects” from the normal underlying tissue and is shed off as a crust.

FIGS. 2 to 8 show photographs 1 to 7 of patients with basal cell carcinomas. The photographs were taken before and after treatment with the topical composition of the present invention.

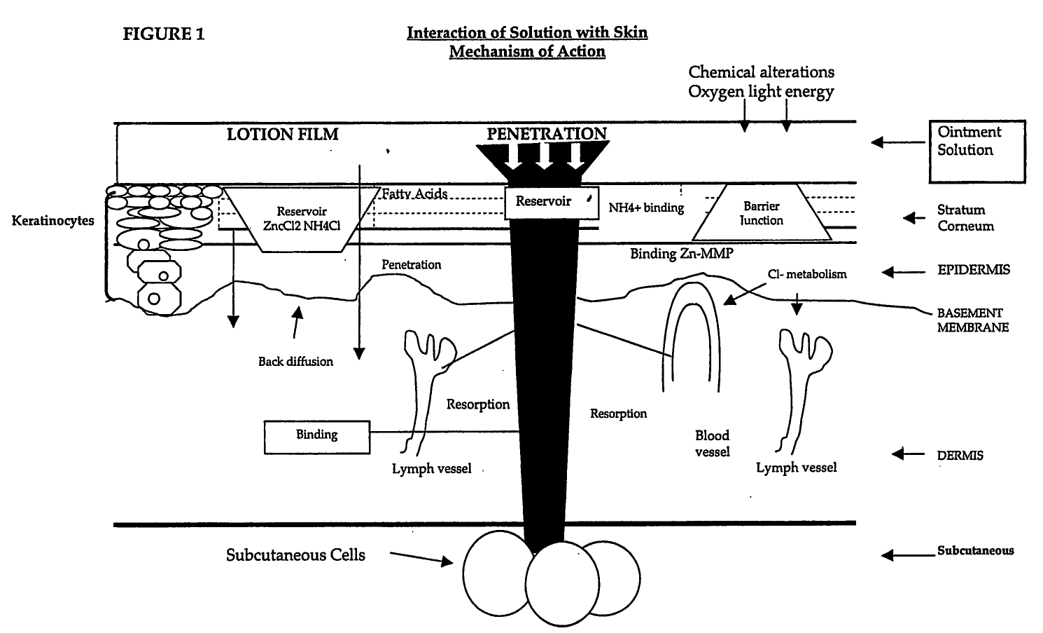

### DETAILED DESCRIPTION OF THE INVENTION

The present invention provides a topical composition that is capable of efficient transcutaneous delivery of an active agent (ie Zn²⁺) to the dermis without the need to apply an external electric current or a chemical reducing agent While not wishing to be bound by theory, this “electrodeless-osmotic delivery” appears to be achieved by the use of small non-metal ions such as Cl⁻ and NH₄⁺ which appear to be efficient in opening up cell membrane channels to allow passage of small metal ions such as Zn²⁺. Another contributing factor appears to be the isoelectric point (pI) of the skin (between 4 and 5), which means that at neutral pH, the cell membranes of skin keratinocytes support a net negative charge. As a consequence, cations are expected to be more efficiently transferred than anions because a negatively charged cell membrane is likely to be permeable to positive ions and repellent of anions. This allows an electrodeless-osmotic flow of solvent in the direction of cation movement into the skin and allows enhanced transcutaneous delivery of Zn²⁺ to the dermis (preferably without penetration of the dermis), since any zinc remaining as zinc ammonium chloride (ie with no net charge) in the solvent, should also be the subject of electrodeless-osmotic flow in the direction of cation movement into the skin.

Once at the dermis, the Zn²⁺ ions are then thought to be able to diffuse into the immediately overlying layers of keratinocytes of the epidermis. In these cells, the Zn²⁺ ions are believed to promote the activity of MMPs to initiate healing of skin lesions and skin cancers. In particular, it appears that the increased activity of MMPs brought about by the binding of Zn2+ ions stimulates, or brings about a resumption of, keratinocyte migration towards the skin surface leading to shedding of damaged and/or apoptotic keratinocyte cells of a skin lesion or skin cancer. In addition, the Zn²⁺ ions disrupt cell/cell interactions which facilitate the shedding of skin.

The present invention provides a topical composition for treatment of a skin disorder, the composition comprising a solution of:
(i) Zn²⁺ ions;
(ii) NH₄⁺ ions;
(iii) Cl⁻ ions; and optionally,
(iv) a suitable excipient and/or carrier.

Preferably, the composition comprises a solution of zinc ammonium chloride and/or zinc chloride, in combination with ammonium chloride and hydrochloric acid.

In a preferred embodiment, the topical composition of the present invention consists of a solution of:
(i) zinc ammonium chloride;
(ii) ammonium chloride;
(iii) hydrochloric acid; and, optionally,
(iv) a suitable excipient and/or carrier.

Zinc ammonium chloride (ZnCl₂.3NH₄Cl) is preferably present in the composition at a concentration in the range of about 30% to 75% weight by volume of the composition, more preferably about 50% to 60% weight by volume of the composition. Most preferably, about 55% by weight by volume of zinc ammonium chloride is used in the composition. However, other suitable amounts outside of these preferred ranges may also be used, as may be readily determined by persons skilled in the art. The composition preferably includes ammonium chloride at a concentration of in the range of about 5% to 20% by weight by volume of the composition, more preferably about 10% by weight by volume of the composition. Preferably, a safe and effective amount of ammonium chloride is used in the composition. The composition preferably includes hydrochloric add at a concentration in the range of about 1% to 5% by weight by volume of the composition, more preferably about 3% to 5% by weight by volume of the composition.

In another preferred embodiment, the topical composition of the present invention consists of a solution of:
(i) zinc chloride;
(ii) ammonium chloride;
(iii) hydrochloric acid; and, optionally,
(iv) a suitable excipient and/or carrier.

Zinc chloride is preferably present in the composition in an amount in the range of about 20% to 60% weight by volume of the composition, more preferably about 30% to 45% weight by volume of the composition. Most preferably, about 35% by weight by volume of zinc chloride is used in the composition. However, other suitable amounts outside of these preferred ranges may also be used, as may be readily determined by persons skilled in the art. The composition preferably indudes ammonium chloride at a concentration in the range of about 5% to 20% by weight by volume of the composition. Preferably, a safe and effective amount of ammonium chloride is used in the composition. Most preferably, the composition includes ammonium chloride at a concentration g in the range of about 8% to 10%o by weight by volume of the composition. The composition preferably indudes hydrochloric add at a concentration in the range of about 1% to 5% by weight by volume of the composition, most preferably 2% to 4% by weight by volume of the composition.

By formulating the Zn²⁺ ions, as provided by zinc ammonium chloride and/or zinc chloride, with other compatible compounds such as ammonium chloride, it has been found that skin irritation caused by the composition is generally low.

NH₄⁺ ions, as provided by ammonium chloride, appears to act as a “membrane fluidiser”. That is, the NH₄⁺ ions appear to act on cell membranes to disrupt contact between the surface and underlying damaged and/or apoptotic keratinocyte cells of damaged skin, and induces short term cellular events such as the release of enzymes responsible for removal of cell debris by macrophages which may lead to the clearance of cellular corpses manifested as a skin lesion.

Cl⁻ ions, as provided by hydrochloric add, is used in the composition to control the pH of the composition and preferably maintain the zinc ammonium chloride and/or zinc chloride and ammonium chloride in a continuous liquid phase (ie to prevent precipitation). In addition, the Cl⁻ ions contribute to the electrodeless-osmotic delivery of the Zn²⁺ ions to the dermis. The composition preferably has a pH of no more than 7, and more preferably a pH in the range of about 2 to 5.

The composition of the present invention preferably contains from about 1 to 99 percent by weight of water. Water is the preferred carrier for the solution of zinc, chloride and ammonium. Preferably, water is used in the range of about 30 to 95 percent by weight by volume of the solution. More preferably, 90 percent by weight by volume of water is used. The composition preferably includes deionised water.

The composition may include an excipient selected from the group consisting of a fragrant agent, emulsifier, surfactant, stabiliser, dye, penetration enhancer, an anti-oxidant and mixtures thereof.

The composition may also include other compatible, active agents suitable for treatment of a range of diseases and conditions, but particularly for the treatment of skin disorders. Additional active agents may therefore include, but are not limited to, anti-infectives such as antibiotics and antiviral agents, analgesics and analgesic combinations, anorexics and appetite suppressants, anthelmintics, anaesthetics, antiarthritics, antiasthma agents, anticonvulsants, antidepressants, antidiabetic agents, antidiarrheals, antihistamines, anti-inflammatory agents, antimigraine preparations, antimotion sickness agents, antinauseants, antineoplastics, antiparkinsonism agents, antipruritics, antipyschotics, antipyretics, antispasmodics, anticholinergics, sympathomimetics, xanthine derivatives, cardiovascular preparations including calcium channel blockers, beta blockers, antiarrhythmics, antihypertensives, diuretics, vasodilators (general, coronary, peripheral and cerebral), central nervous system stimulants, cough and cold relaxants, parasympatholytics, parasympathomimetics, psychostimulants, sedatives, tranquillisers, antioxidants, photoprotective agents, neuropeptides, channel blockers, hormones, vitamins, minerals, other nutrients and herbal extracts or preparations.

The composition can be in the form of a spray, aerosol, lotion, ointment, gel, cream or dispersion and the like. Suitable carriers for a composition in these forms can be selected from water, salt solutions, alcohols, polyethylene glycols, gelatine, lactose, magnesium stearate and silicic acid. The composition may include sterile and non-sterile aqueous solutions. The composition is preferably provided in a soluble form and the zinc ammonium chloride and/or zinc chloride, ammonium chloride and hydrochloric acid are preferably diluted in a soluble sterile buffered saline or water solution.

The composition of the present invention is suitable for treatment of a skin disorder. In the present specification, the term “skin disorder” is to be understood to include diseases and conditions that effect skin, nails or hair. The skin disorder may be associated with an abnormal proliferation of keratinocytes or the abnormal differentiation of epidermal cells to keratinocytes. The skin disorder may be associated with the excessive production of sebum. Examples of skin disorders include, but are not limited to, acne vulgaris, seborrheic dermatitis (also referred to as seborrheic eczema), seborrheic adiposa (also referred to as seborrheic oleosa), seborrheic sicca, psoriasis, eczema, contact dermatitis, irritant dermatitis, ichthyosis and keratosis pilaris. Seborrheic dermatitis is characterised by moderate erythema, dry, moist, or greasy scaling, and yellow crusted patches on various skin areas of the body, including the mid-parts of the face, ears, supraorbital regions, umbilicus, genitalia, and especially the scalp. Seborrheic adiposa is described as oily secretion occurring especially about the nose and forehead. Seborrheic sicca is characterised as dry scaly seborrheic dermatitis. Psoriasis is characterised by scaly, erythematous plaques that may become confluent. Ichthyosis is a non-inflammatory scaling, hyperkeratotic disorder of skin. Keratosis pilaris, or multiple keratin plugs in skin follicles, produces a bumpy appearance to the skin. Hyperkeratosis is common in chronic contact, irritant and atopic (eczema) dermatitis.

The skin disorder may be acne vulgaris, more commonly called acne, which is a common skin disorder affecting a large number of people. Acne can result in physical damage such as scarring or disfigurement. Additionally, acne can cause adverse emotional effects to the individuals afflicted with the condition. Acne results when sebaceous follicles, located primarily on the face and trunk, become obstructed with sebum and epithelial cells. Sebum is produced by sebaceous glands in the follicles and epithelial cells are desquamated from the walls of the follicles. The sebum and the desquamated epithelial cells obstruct the sebaceous follicles. Obstruction of the follicles creates microcomedones which may evolve into comedones (non-inflammatory lesions, eg open and dosed comedones such as whiteheads and blackheads) or inflammatory lesions (eg inflammatory nodules, pustules and papules). A residing anaerobic bacterium, Propionibacterium acnes (P. acnes) proliferates in this environment of excessive sebum and follicular cells and may produce localised inflammation. Acne can be primary (idiopathic) or secondary (due, for example, to the application of cosmetics). Included in the definition of acne for the purposes of the present invention are cosmetically undesirable skin conditions commonly referred to as pimples, blemishes, skin imperfections, etc.

The skin disorder may be due to hypertrophy of the stratum corneum, an occurrence also described as hyperkeratinisation. The thickened superficial layer of the epidermis results in scale-like plaques on the surface of the skin. These scaly plaques are the manifestation of a group of disorders termed ichthyoses because of their resemblance to fish scales. The plaques may be symptoms of a skin disorder and accordingly prohibit the treatment of these disorders originating in underlying layers of the skin. The hypertrophied skin layer may also harbour infections within itself. Typical examples of ichthyoses include psoriasis, pityriases, rosacea, and seborrheic dermatitis. Dermaphytoses are ichthyoses caused by fungal infections. The hyphae and spores are confined to nonviable portions of tissue and thus proliferate in the hyperkeratinised tissues of skin, hair, and nails. Examples of typical dermaphytoses include tinea capitis (cradle cap), tinea pedis (athlete's foot), and tinea anguium. The skin disorder may include corns and warts.

The skin disorder may include inflammation of the skin. The terms eczema and dermatitis are generally used names for severe inflammation of the skin, usually with redness, swelling, oozing, rusting or scaling of lesions which are usually itchy. Eczema may take the form of contact dermatitis (due to skin contact with the cause) or atopic dermatitis in individuals who are “atopic” or allergic by nature. If the scalp is involved, the disorder is known as seborrheic dermatitis. Dermatitis can be caused by chemicals, plants, shoes, clothing, metal compounds and even medicines used to treat dermatitis. In atopic dermatitis, environmental temperature, humidity changes, bacterial skin infections, airborne allergens and garments (eg wool), may all bring about dermatitis.

The present invention also provides a method for treating or preventing a skin disorder in a subject, the method comprising applying to the skin of the subject an effective amount of a topical composition according to the first or second aspect.

In the method of the present invention, the term “effective amount” means a suitable amount of the topical composition including an effective concentration of the selected compounds of the composition sufficient to provide treatment or prevention of a skin disorder in a subject. The effective amount of the composition used in the method of the present invention may vary depending on the subject and the type and level of the skin disorder. The effective concentration of each compound used in the composition is subject to the degree to which penetration enhancement is achieved. For example, when the increase in penetration is relatively large, lesser amounts of the zinc ammonium chloride and/or zinc chloride can be used.

The magnitude of a therapeutic dose of the composition in the acute or chronic management of skin disorders will vary with the severity of the disorder to be treated. The dose and dose frequency will vary according to the response of the subject. The term “unit dose” is meant to describe a single dose although a unit dose may be divided, if desired. About 1 to 2 unit doses of the present invention are typically administered per day, preferably 1 dose per day every 24 hour interval. Topical administration is generally preferred for the composition and method of the invention.

The subject treated by the method of the invention may be selected from, but is not limited to, the group consisting of humans, sheep, cattle, horses, bovine, pigs, poultry, dogs and cats.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention will hereinafter be described by way of the following non-limiting Examples.

### EXAMPLE 1

### Topical Composition Comprising Zinc Chloride

| Part | | |
|---|---|---|
| A | Deionised water 800 ml | |
| | Zinc chloride 450 grams/L | granular (endotoxin tested) |
| | Ammonium chloride 110 grams/L | granular |
| B | Hydrochloric acid 40 grams/L | concentrated HCl (0.1) ml |
| C | Phenolic fragrance 2 grams/L | flower extract in 85% ethyl |
| | | alcohol |

Deionised water was measured in the volume of 800 ml and poured into a processing tank with a speed regulated stirrer. The water was heated to about 60° C. The stirrer was subsequently begun at low speed and maintained throughout the dissolution process until all solutes had been dissolved and the liquid became dear. Zinc chloride and ammonium chloride granules were weighed and sprinkled in and mixed until a clear and uniform liquid was obtained. The mixture was then cooled to room temperature while the stirring continued. The hydrochloric acid was then added very slowly. Fractions of the solution were taken from different sites of the tank to measure the pH of the solution. Only after a uniform pH was obtained was the phenolic fragrance added. Stirring of the solution was continued with reduced speed until a completely clear liquid was obtained. The solution was then made up to a final volume of 1 litre with deionised water. The pH at 25° C. was between 2 and 4 when measured with a pH strip indicator.

### EXAMPLE 2

### Topical Composition Comprising Zinc Chloride

| Part | | |
|---|---|---|
| A | Deionised water 900 ml | |
| | Zinc chloride 225 grams/L | granular (endotoxin tested) |
| | Ammonium chloride 55 grams/L | granular |
| B | Hydrochloric acid 20 grams/L | concentrated HCl (0.05) ml |
| C | Phenolic fragrance 3 grams/L | flower extract in 85% ethyl |
| | | alcohol |

Deionised water was measured in the volume of 900 ml and poured into a processing tank with a speed regulated stirrer. The water was heated to 60° C. Part A was added to the tank and stirred until all solids had dissolved and a clear solution was obtained. The temperature of the solution was reduced to room temperature. Part B was added very slowly to the tank while the solution was kept stirring at low constant speed. Fractions of the solution were taken from different sites of the tank and tested for uniformity of pH. After a uniform pH was obtained, phenolic fragrance in the volume of 1 ml was added with constant stirring. The pH of the solution was checked again as mentioned above. If the pH had to be adjusted, it was done by adding drops of 0.1N HCl. The final volume of the composition of 1 litre was made up with deionised water. The pH at 25° C. was between 2 and 4 when measured with a pH strip indicator.

### EXAMPLE 3

### Topical Composition Comprising Zinc Chloride

| Part | | |
|---|---|---|
| A | Deionised water 900 ml | |
| | Zinc chloride 337.5 grams/L | |
| | Ammonium chloride 82.5 grams/L | |
| B | Hydrochloric acid 30 grams/L | concentrated HCl (0.75) ml |
| C | Phenolic fragrance 1 gram/L | flower extract in 85% |
| | | alcohol (1 ml) |
| D | Euginia aromatica 1 ml | flower bud extract in 85% |
| | | alcohol (0.5 ml) |
| | Commiphora abysinica 1 ml | oleo gum resin in 65% |
| | | alcohol (0.5 ml) |

The method of preparation followed was the same as that above (ie examples 1 and 2) for Part A, Part B and Part C. After Part C was mixed well for about 10 minutes, Part D was added. The final volume of the composition was made to up to 1 litre with deionised water.

### EXAMPLE 4

### Treatment Regimen Study

The compositions described in examples 1-3 can be used in three separate groups by applying, as a lotion, to actinic keratosis, basal cell carcinoma, and nevoid basal cell carcinoma lesions on human skin. Everyday, selected study subjects should apply (at about the same time of each day) the lotion by dipping a cotton tipped stick into the lotion and rolling the cotton tip lightly over the lesion. The subjects should then wait for the solution to dry from the skin surface without washing or wiping for at least 4 hours. Evaluation should be done everyday for total lesions present and overall appearance of the lesions.

Testing of the Treatment Regimen

The composition prepared according to examples 1-3 should each be administered to 15 subjects who exhibit a Grade 2-4 skin condition according to the grading scale provided below:

0: Facial and arm skin need not be perfectly clear. A few scattered actinic keratosis or nevoid or basal cell carcinoma may be present, but these should be visible only on very dose examination.

2: About 1/16^{th} of facial or arm area is involved, with the disorders. A few small or large prominent blebs may be present.

4: About ⅛^{th} facial and arm area is involved, with small and large blebs are usually present. If lesions are large, subject may have Grade 4 severity, although less than half of skin area is involved.

On the first day of the study, all subjects should be acclimatised to ambient temperature and relative humidity for 10 minutes. After the equilibration period, a trained technician should examine each subject's face for traces of other cosmetics and record the number of lesions in each of 4 sections of the face and arms. The lesions of the 4 sections should be totalled to obtain a global assessment score for each subject. Clinical photographs should be taken in various poses for each subject. Subjects should be provided with the treatment lotion and given the following instructions for treatment:

Wash skin to be treated with soap and water and pat dry. Apply twice per day (once in the morning and once in the evening). Dip the cotton tip in the composition and roll over the affected skin. Let the skin surface dry out. Do not wash or apply any other material for at least 4 hours. Another treatment regimen should involve only one application per day.

Subjects should be not be permitted to use their customary make up products during the study and should also be instructed not to introduce any new facial treatment products during the study. At the end of the first week of the test period, subjects should be evaluated for an interim count of total lesions and clinical photographs. After two weeks, subjects should return for a final lesion count. Standard paired t-tests should be used to determine statistically significant differences between baseline and week 1 and week 2 total facial lesion counts. Statistical significance should exist for all p-values less than or equal to 0.05 at 95% confidence level. Improvement scores for the appearance of the skin blebs in clinical photographs should be analysed using Z-tests.

Total Lesion Count Following Treatment Regimen

The blebs present on the skin of each subject should be evaluated by visual examination using the grading scale described herein. The number of lesions visible on the face should be counted at each visit and the number recorded. A global assessment score (ie the total of all lesions), should be recorded for each visit. Reductions in the global assessment score are indicative of a reduced incidence and/or severity of lesions. A sample data for total lesion count is provided below

Total Lesion Count

| | Day 0 | Week 1 | Week 2 |
|---|---|---|---|
| Mean | 20 | 10 | 4 |
| Mean Percent Difference from Day 0 | | | |
| | | 5 | 1 |

The treatment should show a statistically significant decrease in the number of lesions when compared to the day 0 values. The p value should be <0.01.

Photographic Evaluation Following Treatment Regimen

Photographs or subjects should be taken during designated visits using a Canfield Clinical System of imaging equipment. This particular system permits comparison of photographs to be made with the confidence that the only factors which may have changed are those resulting from treatment This is achieved by precisely and reproducibly positioning the head of the subject and carefully controlling the lighting, film type and processing. Photographs should be visually assessed and evaluated by a trained technician before and after use of the composition. The following scoring scale should be used for visual assessment of the skin:

1=no improvement

2=slight improvement

3=mild improvement

4=moderate improvement

5=extreme improvement

Improvement scores for the appearance of skin blebs in clinical photographs should be analysed using Z-tests. At the start of the regimen, all subjects should be photographed. For the week 1 and week 2 scores, the number of subjects exhibiting improvements scoring a two (2), three (3), four (4) or five (5) should be compared to the number of subjects exhibiting no improvement, scored as a one (1). A sample data for an improvement assessment of the overall appearance of blebs, rated from clinical photographs, is provided below.

Photographic Evaluation

| Score | 1 | 2 | 3 | 4 | 5 |
|---|---|---|---|---|---|
| At start | 10 | 0 | 0 | 0 | 0 |
| Number of subjects | | | | | |
| Week 1 | 0 | 1 | 4 | 5 | 0 |
| Number of subjects | | | | | |
| Assigned each Score | | | | | |
| Percentage | a % | b % | | | |
| Z-score | −x | | | | |
| Week 2 | 0 | 0 | 0 | 2 | 8 |
| Number of subjects | | | | | |
| Assigned each Score | | | | | |
| Percentage | >a % | >b % | | | |
| Z-score | >−x | | | | |

The number of subjects exhibiting improvement from baseline in the overall appearance of blebs at week 1 should be greater than subjects with no improvement. The Z-score obtained at week 1 should correspond to improved skin appearance having a statistical significance at about 75% confidence level. In the week 2 photographs, the number of subjects exhibiting improvement from baseline in the overall appearance of blebs should be greater than subjects with no improvement. The Z-score obtained at week 2 should correspond to improved skin appearance having statistical significance at a 95% or more confidence level.

### EXAMPLE 5

### Treatment of Patients With a Topical Composition Comprising Zinc Chloride

Female Caucasian Patient

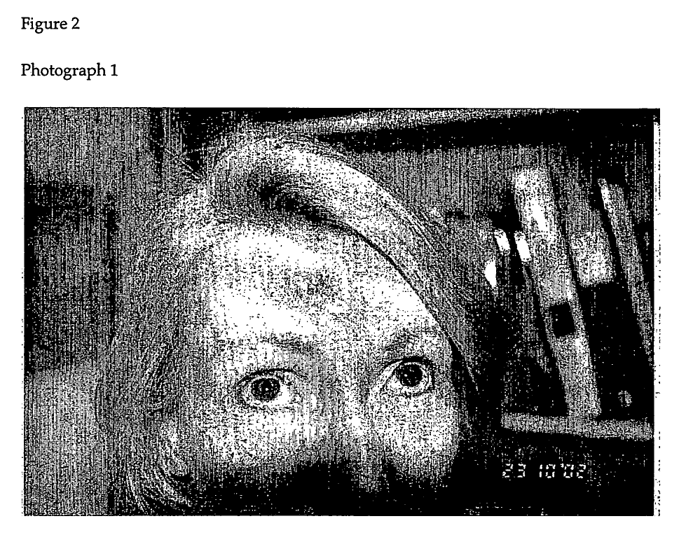

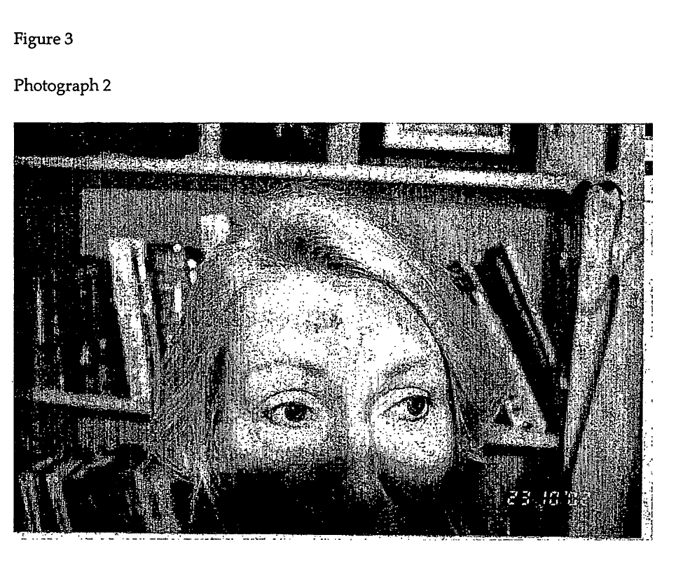

The first subject tested was a 57 year old, female suffering from an extremely large well-embedded basal cell carcinoma in the middle of the forehead. The carcinoma was ranging from 3 cms to 2.5 cms in size and had 4 separate protrusions at its outer edge. After thoroughly washing with soap and water and then drying the surface to which the lotion was to be applied to remove all residue of any make-up or ointments, a single application of the lotion described in Example 3 was made directly to the basal cell carcinoma (BCC). This was termed as day 1 of the treatment. After the lotion had been applied with a sterile applicator (cotton-tipped), it was allowed to dry on the patient's forehead. The subject was asked to keep it uncovered and not apply any detergent or other chemicals until advised during the treatment period Photograph 1 shown in FIG. 2 was taken prior to application of the lotion on day 1. The photograph show s different angles of the BCC on the patient's forehead. There were four separate protrusions on the BCC located at approximately 3, 6, 9 and 12 o'clock positions on the BCC. These protrusions are termed #1, #2, #3 and #4 respectively. One hour after the lotion application on day 1 the patient exhibited a localised reaction across the BCC site. There was a colour change across most of the surface of the BCC site from flesh pink/yellow colour to a pink/red colour at the extremities and in the adjoining surface skin and to a white coloured surface across most of the BCC site. The whitish colour change was accompanied by the formation of a soft crust formation on the surface of the BCC site. The whitish central portion began to show signs of dehydration and wrinkling of the skin. Photograph 2 of FIG. 3 shows the 1 hour effect of the single lotion application.

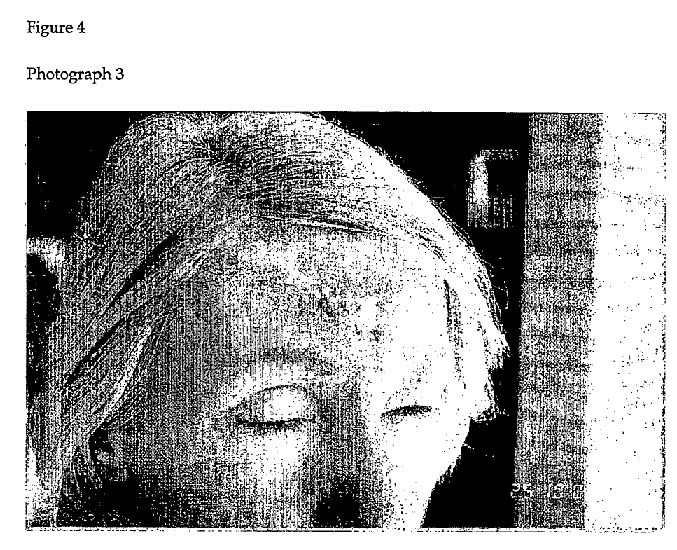

During the next 14 days the initial localised reaction underwent further changes. The soft whitish surface crust described in the 1 hour effect of the single application penetrated deeper into the BCC and progressively changed from a soft surface to a hard crusty scab extending beneath the surface. There was a change in colour at the periphery and edges of the BCC site from pink/red including in the adjoining surface skin area. Progressively the redness disappeared in the adjoining surface skin area and on the periphery of the BCC and there was a shrinking of the hard surface scab leading to a complete healing over of all of the BCC site with the exception of #3 protrusion. The entire process resulted in the disappearance of 3 of the BCC protrusions (those seen at #1, #2 and #4 in the photograph), leaving the fourth #3 protrusion of the tumour less reactive as shown in photograph 1 of FIG. 2. However, compared to day 1, protrusion #3 was significantly reduced in size indicating the action of the lotion to be slow (see photograph 3 of FIG. 4).

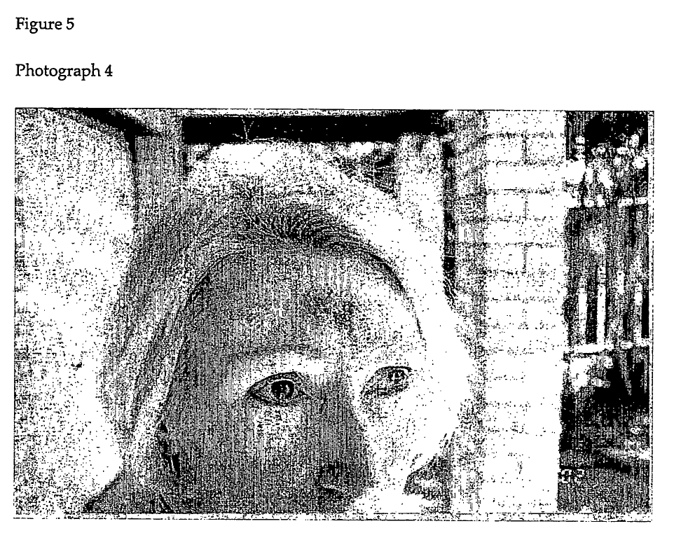

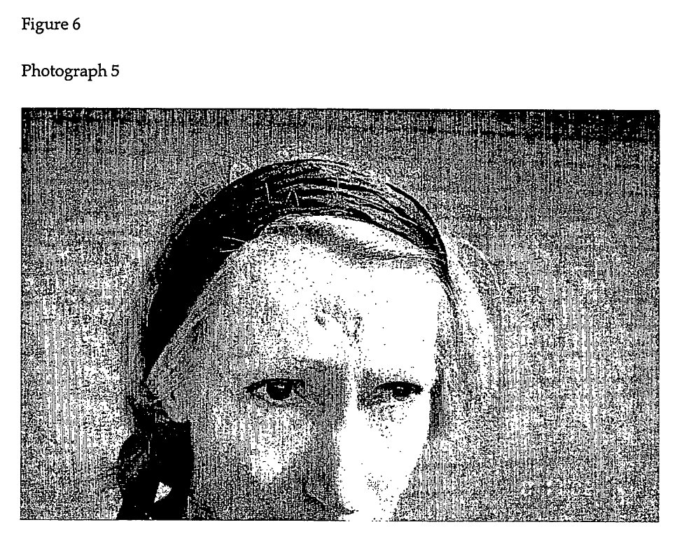

The crust formation initiated on day 3 is shown in photograph 4 of FIG. 5. The central whitish portion of the carcinoma began showing signs of apoptosis and cell death with the formation of wrinkling and early signs of drying. The day 8 progression of the crust to a scab-like form across the entire BCC is shown in photograph 5 of FIG. 6. The day 17 progression of the healing process leading to the absence of protrusions #1, #2 and #4 and the blending of the former BCC site surface into normal surrounding skin, except for protrusion #3, is shown in photograph 3 of FIG. 4.

On day 17 a second application of the lotion was administered to the remaining somewhat active #3 BCC tumour site on the forehead of the subject. The whitish central portion had progressed from a wrinkled shrinking state on day 1 to a hard scab on day 8 which was attached to the base of the carcinoma and by day 17 the scab which previously extended from the centre to the peripheral area involving the #1, #2 and #4 protrusions had completely vanished. However protrusion #3 was still present but reduced in size and pinkish in colour. It appears that protrusion #3 may have been at a different phase of growth or had a different concentration of zinc receptors from those of the other three as it was responsive but failed to progress into cell death (see photograph 3 of FIG. 4). The preparation and application methodology was identical to the preparation and application methodology undertaken on day 1.

Male Caucasian Patient

The second patient was a male suffering from 4 BCC formations on the balding scalp of his upper skull at three separate locations. A single application of the lotion described in Example 3 was applied to the three separate locations on the scalp of the subject. Prior to application of the lotion the scalp of the patient was thoroughly washed and dried. The lotion was applied with a sterile applicator and was allowed to dry on the patient's scalp. At each of the application sites there was a small BCC approximately 5 cm and at the third site there was a contiguous second BCC the approximate size of a pinhead. The lotion was applied on day 1 of the treatment. One hour after the lotion application on day 1 the patient exhibited a localised reaction across the entire surface of each BCC at the 3 separate sites. There was a colour change from flesh pink to pink/red at the extremities and in the adjoining surface skin and to white across the BCC site. The whitish parts developed a soft surface crust. Progressively over the succeeding days the whitish surface crust penetrated below the surface and turned into a scab-like crust which then slowly shrank and healed over across the entire BCC site with healing being demonstrated by the complete absence of any tumour and the skin reverting to the colour and texture of the surrounding skin. By day 8 the 4 BCCs had completely disappeared from the patient's scalp.

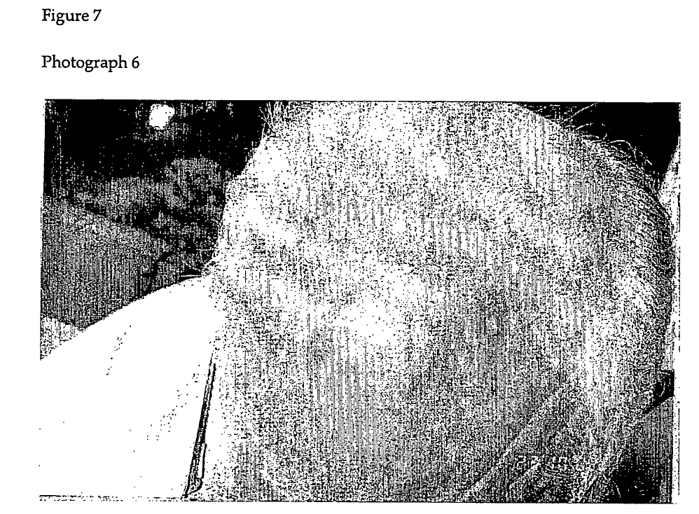

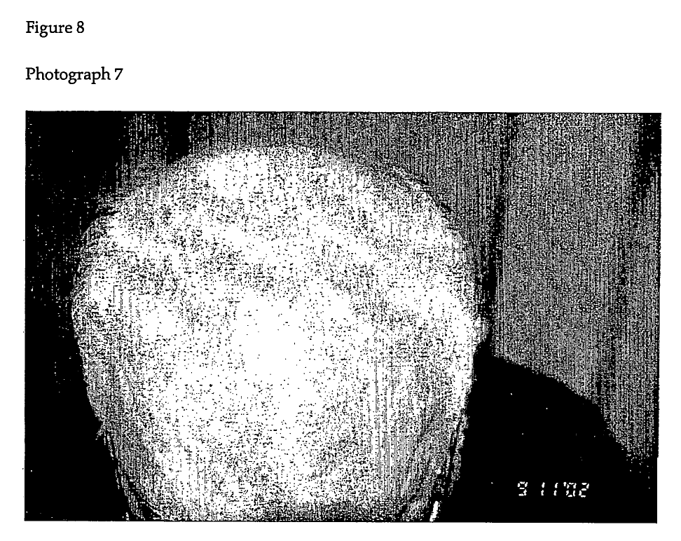

The day 2 localised reaction and crusty formation on the scalp after the single application of the lotion is shown in photograph 6 of FIG. 7. The day 10 and day 19 complete elimination of the 4 BCC tumours from the patient's scalp after the single application of the lotion is shown in photograph 7 of FIG. 8.

### EXAMPLE 6

### Topical Composition Comprising Zinc Ammonium Chloride

| Part | | |
|---|---|---|
| A | Deionised water 800 ml | |
| | Zinc ammonium chloride 550 | granular (endotoxin tested) |
| | grams/L | |
| B | Hydrochloric acid 40 grams/L | concentrated HCl (0.1) ml |
| C | Ammonium chloride 100 grams/L | granular |

The composition above was prepared by firstly measuring deionised water in the volume of 800 ml and pouring it into a processing tank with a speed regulated stirrer. The water was heated to about 60° C. The stirrer was subsequently begun at low speed and maintained throughout the dissolution process until all solutes had been dissolved and the liquid became clear. Zinc ammonium chloride was weighed and sprinkled in and mixed until a clear and uniformed liquid had been obtained. Hydrochloric acid was measured and then slowly dripped into the mixture. The ammonium chloride granules were weighed and sprinkled in and added very slowly until fully dissolved. Stirring of the solution was continued with reduced speed until a completely clear liquid was obtained. The solution was then made up to a final volume of 1 litre with deionised water and allowed to cool to room temperature.

### EXAMPLE 7

### Treatment of Patients With a Topical Composition Comprising Zinc Ammonium Chloride

(a) Female Caucasian Patient

The subject tested was a 55 year old female who had a nose reconstruction for medical reasons. A post-operative checkup of the subject by a surgeon identified a possible basal cell carcinoma (BCC) or squamous cell carcinoma (SCC) on the left tip of the subject's nose. The carcinoma was approximately 4 mm in diameter on the surface of the subject's nose. A punch biopsy of the carcinoma measuring 1 mm in diameter and 5 mm in thickness was taken by a specialist pathologist. The written report by the pathologist indicated that the microscopic examination of the carcinoma showed solar keratosis with microinvasive carcinoma, more suggestive in appearance of SCC than BCC. The report also noted that there was marked actinic elastosis of dermal collagen.

The carcinoma on the skin of the subject was treated by firstly thoroughly washing with soap and water and then drying the surface of the affected skin and all residues of any make-up or ointments was substantially removed. This was followed by a single application of the composition described in Example 6 directly to the squamous cell carcinoma (SCC). This was termed as day 1 of the treatment. After the composition had been applied with a sterile applicator (cotton-tipped), it was allowed to dry on the subject's skin. The subject was asked to keep the treated skin uncovered and to avoid contacting the skin with any detergents or other chemicals during the treatment period. Shortly after the topical composition application on day 1, the subject exhibited a localised reaction across the SCC site. There was an observable colour change across most of the surface of the SCC site from flesh pink colour to a white coloured surface. The whitish colour change was accompanied by the formation of a soft crust formation on the surface of the SCC site. On days 2 and 3, the colour of the treated SCC site changed from whitish to more yellow and on day 4 it formed a thicker surface scab crust which gradually hardened from day 4 onwards. On day 7, the scab had fallen off revealing a reddish coloured surface similar in appearance to the initial appearance of the SCC. Also on day 7, the composition was reapplied with a sterile applicator and allowed to dry. Shortly after its reapplication the subject exhibited a localised reaction across the treated SCC site, initially whitish in colour followed on days 8 and 9 by the formation of a soft yellowish crust across the surface, which by day 11, had hardened into a surface scab thicker on this occasion than the first application. On day 16, the surface scab fell off leaving clean and clear skin slightly more pinkish than the areas immediately adjacent to it Over the course of days 17 to 20, the pinkish colour faded at the site of the lesion and normal skin colour returned. There was no evidence of the existence of the SCC by day 20.

After about two months after the initial skin biopsy, a further punch biopsy was performed by the same pathologist who had performed the initial biopsy. The skin biopsy consisted of a punch biopsy measuring 2 mm in diameter and 3 mm in thickness. The written report stated that on microscopic examination there was no evidence of either intraepidermal or invasive malignancy at the multiple levels examined. As in the initial biopsy, the pathologist reported sections showed sun damaged skin with severe actinic elastosis of dermal collagen.

(b) Male Caucasian Patient

The second subject tested was a 65-year-old male who had undergone renal transplant in 1988 with consequent long-term anti-immune therapy. The subject had a history of multiple basal cell carcinoma (BCC) or squamous cell carcinoma (SCC) on the face, neck, trunks arms, hands and legs which had resulted in surgical excision on at least ten separate occasions between 1989 and 1997 including, on two occasions, admission to hospital. The hospital medical records confirmed the removal of thirty-eight SCC and BCC by histopathology in most cases and on two occasions, skin grafts were undertaken. The subject also reported that there had been excisions of an additional 20 BCC and SCC undertaken, however no medical records were available to confirm the additional excisions.

Prior to treatment with the topical composition, the subject had multiple major BCCs on and adjoining the left ear, multiple BCCs and SCCs to the left temple and the left cheek, multiple BCCs and SCCs on the left arm and hand, several large SCCs on the nose, multiple BCCs on the right temple and cheek and multiple BCCs and SCCs on the right arm and hand. The subject reported that his skin cancer specialist had recommended the removal of his left ear, skin grafts to the left cheek, left and right arms and a surgical reconstruction of his nose. Having regard to the severity of the condition, the multiplicity of lesions at all affected areas, the size of the major lesions and the number of sites to be treated, it was apparent that the subject would require multiple applications of the composition. However, the proposed treatment was to be initially confined to several sites and then progressively extended to other sites if the lesions initially treated responded positively to the application of the composition.

The treatment regime consisted of thoroughly washing with soap and water and then drying the skin surface to which the composition was to be applied to remove all residues of any make-up or ointments and dirt and grease. The composition as described in Example 6 was applied on each occasion with a sterile applicator and was allowed to dry on the lesion site. The subject was asked to keep the treated skin uncovered and to avoid contacting the skin with any detergents or other chemicals during the treatment period. However, the subject's occupation was a plant operator who was required to shower and wash himself each day to remove the dirt and grease which collected during his work. Accordingly, the composition was reapplied every second or third day. Additionally, on several occasions, prior to a reapplication of the composition, the lesion sites were cleansed with a diluted hydrogen peroxide solution to assist removal of dirt and oil to provide a clean surface onto which the next application of composition could be made.

Following treatment with the topical composition, the patient sustained a localised reaction across each lesion site within two hours of each application of the composition. Each of the treated lesion sites showed a colour change across the surface from a flesh pink/yellow colour to a white coloured surface accompanied by the formation of a soft crust formation which progressively hardened and changed from a whitish to more yellow colour then forming a thicker surface scab crust which gradually hardened and, on most occasions, either fell off on its own account (or was inadvertently knocked off by the patient during his daily occupation or whilst cleansing his skin at the end of his daily work).

Small lesions (ie less than 2 mm in diameter) immediately responded to treatment and disappeared within 7 days of the first application of the composition (in each case, there was a subsequent reapplication of the composition made to the surface irrespective of the physical observations). The medium lesions (ie between 2 mm and 5 mm in diameter) also responded to the application of the composition and exhibited the reactions described above. In most cases, the medium lesions were cured by day 14 after the first application of the composition (again, in all cases, there were subsequent reapplications of the composition irrespective of the physical observation made subsequent to the first application). The large lesions (ie greater than 5 mm in diameter) responded progressively to the subsequent reapplications of the treatment and exhibited the reactions described above. The large SCC on the left arm and right hand of the subject required approximately ten reapplications of the composition every second or third day after the initial application before each of these lesions responded fully to the treatment regime. In the period of about four months, there was a complete response to the application of the composition and a cure of all lesions present at the time of the initial consultation. After about four months after the initial treatment, there were no new lesions identified on the subject's body nor at the lesion sites initially present.

### EXAMPLE 8

### Topical Composition Comprising Zinc Chloride

| Part | | |
|---|---|---|
| A | Deioinised water 300 ml | |
| | Zinc Chloride 300 gms/L | granular (98% pure) |
| | Ammonium Chloride 103.34 gms/L | granular |
| B | Hydrochloric acid 300 ml | (0.2% strength) |

The composition was prepared by following the steps outlined below:
1. Weighed out 300 grams ZnCl₂ powder and transferred into a conical glass flask.
2. Weighed out 103.34 grams NH₄Cl powder and transferred into the same conical glass flask into which ZnCl₂ is added.
3. Measured 300 ml deionised water and poured into the flask.
4. The flask was kept on a magnetic stirrer.
5. The solid contents of the flask was dissolved with medium stirring without heat at room temperature.
6. When no solids were present in the flask 300 ml of the 0.2% HCL was poured into the flask.
7. Stirring was continued for 15 minutes.
8. pH of the mixture was checked with pH strip. The pH indicated was 4.
9. 37% HCl was added drop by drop and the mixture was allowed to stir for 10 minutes.
10. pH was checked
11. More additions of 37% HCl was done with 10 minute intervals until a final pH of 2 was obtained.
12. The final volume of the composition was made up to 1000 ml by addition of distilled water.

The topical composition was prepared by dissolving the salts together in water. Low strength HCl (0.2%) was added to the salt solution and allowed to stir for a while. This was done to allow the dissociation to be formed slowly due to the low acidic medium. Finally the pH was brought down by minute amount of concentrated HCl. This allows the ions to be in free form in the solution. In addition, in use the composition may also minimise the amount of water molecules that hydrates the basement membrane of BCCs thereby increasing the formation of the zinc layer. The application of the composition onto a lesion would then result in the formation of a scab and the shedding of dead keratinocytes from the surface of the skin.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

1. A topical composition for treatment of a skin disorder, the composition comprising a solution of:
(i) ZN²⁺ ions;
(ii) NH₄⁺ ions;
(iii) Cl⁻ ions; and optionally,
(iv) a suitable excipient and/or carrier.

2. A topical composition according to claim 1, wherein the composition comprises a solution of zinc ammonium chloride and/or zinc chloride, in combination with ammonium chloride and hydrochloric acid.

3. A topical composition for treatment of a skin disorder, the composition consisting of a solution of:
(i) zinc ammonium chloride;
(ii) ammonium chloride;
(iii) hydrochloric acid; and optionally;
(iv) a suitable excipient and/or carrier.

4. A composition according to claim 3, wherein the solution includes zinc ammonium chloride at a concentration in the range of about 30% to 75% by weight by volume of the composition.

5. A composition according to claim 3, wherein the solution includes zinc ammonium chloride at a concentration of about 55% by weight by volume of the composition.

6. A composition according to claim 3, wherein the solution includes ammonium chloride at a concentration in the range of about 5% to 20% by weight by volume of the composition.

7. A composition according to claim 3, wherein the solution includes ammonium chloride at a concentration of about 10% by weight by volume of the composition.

8. A composition according to claim 3, wherein the solution includes hydrochloric acid at a concentration in the range of about 1% to 5% by weight by volume of the composition.

9. A composition according to claim 3, wherein the solution includes hydrochloric acid at a concentration in the range of about 3% to 5% by weight by volume of the composition

10. A topical composition for treatment of a skin disorder, the composition consisting of a solution of:
(i) zinc chloride;
(ii) ammonium chloride;
(iii) hydrochloric acid; and, optionally,
(iv) a suitable excipient and/or carrier.

11. A composition according to claim 10, wherein the solution includes zinc chloride at a concentration in the range of about 20% to 60% by weight by volume of the composition.

12. A composition according to claim 10, wherein the solution includes zinc chloride at a concentration in the range of about 30% to 45% by weight by volume of the composition.

13. A composition according to claim 10, wherein the solution includes ammonium chloride at a concentration in the range of about 5% to 20% by weight by volume of the composition.

14. A composition according to claim 10, wherein the solution includes ammonium chloride at a concentration in the range of about 8% to 10% by weight by volume of the composition.

15. A composition according to claim 10, wherein the solution includes hydrochloric acid at a concentration in the range of about 1% to 5% by weight by volume of the composition.

16. A composition according to claim 10, wherein the solution includes hydrochloric acid at a concentration in the range of about 2% to 4% by weight by volume of the composition.

17. A composition according to claim 1, wherein the solution is an aqueous solution.

18. A composition according to claim 1, wherein the solution has a pH of no more than about 7.

19. A composition according claim 1, wherein the solution has a pH in the range of about 2 to 5.

20. A composition according to claim 1, wherein said carrier comprises deionised water.

21. A composition according to claim 1, wherein the composition includes water at about 1% to 99% by weight by volume of the composition.

22. A composition according to claim 1, wherein the composition includes water at about 30% to 95% by weight by volume of the composition.

23. A composition according to claim 1, wherein the composition includes an excipient selected from the group consisting of a fragrant agent, surfactant, stabiliser, dye, penetration enhancer and anti-oxidant.

24. A composition according to any one claim 1, wherein the composition is in the form of a spray, aerosol, lotion, ointment, gel, cream or dispersion and the like.

25. A method for treating a skin disorder in a subject, the method comprising applying to the skin of the subject an effective amount of a topical composition according to claim 1.

26. A method according to claim 25, wherein the skin disorder is selected from the group consisting of skin lesion, hyperproliferative skin disorder, inflammatory skin disorder, actinic keratosis, solar lentigines, psoriasis, dermatitis, eczema, tenia, melanoma, basal cell carcinoma and squamous cell carcinoma.

27. A composition according to claim 3, wherein the solution is an aqueous solution.

28. A composition according to claim 10, wherein the solution is an aqueous solution.

29. A composition according to claim 3, wherein the solution has a pH of no more than about 7.

30. A composition according to claim 10 wherein the solution has a pH of no more than about 7.

31. A composition according to claim 3, wherein the solution has a pH in the range of about 2 to 5.

32. A composition according to claim 10, wherein the solution has a pH in the range of about 2 to 5.

33. A composition according to claim 3, wherein said carrier comprises deionised water.

34. A composition according to claim 10, wherein said carrier comprises deionised water.

35. A composition according to claim 3, wherein the composition includes water at about 1% to 99% by weight by volume of the composition.

36. A composition according to claim 10, wherein the composition includes water at about 1% to 99% by weight by volume of the composition.

37. A composition according to claim 3, wherein the composition includes water at about 30% to 95% by weight by volume of the composition.

38. A composition according to claim 10, wherein the composition includes water at about 30% to 95% by weight by volume of the composition.

39. A composition according to claim 3, wherein the composition includes an excipient selected from the group consisting of a fragrant agent, surfactant, stabiliser, dye, penetration enhancer and anti-oxidant.

40. A composition according to claim 10, wherein the composition includes an excipient selected from the group consisting of a fragrant agent, surfactant, stabiliser, dye, penetration enhancer and anti-oxidant.

41. A composition according to claim 3, wherein the composition is in the form of a spray, aerosol, lotion, ointment, gel, cream or dispersion and the like.

42. A composition according to claim 10, wherein the composition is in the form of a spray, aerosol, lotion, ointment, gel, cream or dispersion and the like.

43. A method for treating a skin disorder in a subject, the method comprising applying to the skin of the subject an effective amount of a topical composition according to claim 3.

44. A method for treating a skin disorder in a subject, the method comprising applying to the skin of the subject an effective amount of a topical composition according to claim 10.

45. A method according to claim 43, wherein the skin disorder is selected from the group consisting of skin lesion, hyperproliferative skin disorder, inflammatory skin disorder, actinic keratosis, solar lentigines, psoriasis, dermatitis, eczema, tenia, melanoma, basal cell carcinoma and squamous cell carcinoma.

46. A method according to claim 44, wherein the skin disorder is selected from the group consisting of skin lesion, hyperproliferative skin disorder, inflammatory skin disorder, actinic keratosis, solar lentigines, psoriasis, dermatitis, eczema, tenia, melanoma, basal cell carcinoma and squamous cell carcinoma.
